(19) United States
(12) Patent Application Publication
(10) Pub. No.: US 2013/0203184 A1
(43) Pub. Date: Aug. 8, 2013

(54) METHOD FOR DETECTION OF CLEAVAGE PRODUCT OF SOLUBLE AMYLOID-B PRECURSOR PROTEIN 770B FOR DIAGNOSIS OF DISEASES ASSOCIATED WITH ACCUMULATION OF AMYLOID-B PEPTIDE

(71) Applicants: Shinobu Kitazume, Saitama (JP); Naoyuki Taniguchi, Saitama (JP); Yuriko Tachida, Saitama (JP); Takaomi Saido, Saitama (JP); Yasuhiro Hashimoto, Fukushima (JP); Takashi Honda, Fukushima (JP)

(72) Inventors: Shinobu Kitazume, Saitama (JP); Naoyuki Taniguchi, Saitama (JP); Yuriko Tachida, Saitama (JP); Takaomi Saido, Saitama (JP); Yasuhiro Hashimoto, Fukushima (JP); Takashi Honda, Fukushima (JP)

(73) Assignee: RIKEN, Wako-shi, Saitama (JP)

(21) Appl. No.: 13/813,073

(22) PCT Filed: Jul. 29, 2011
(86) PCT No.: PCT/JP2011/067530

§ 371 Date: Apr. 15, 2013

(30) Foreign Application Priority Data
Jul. 29, 2010 (JP) 2010-171122

(51) Int. Cl. G01N 33/68

(52) U.S. Cl. CPC G01N 33/6896
USPC 436/501

(57) ABSTRACT

The present invention provides a method of detecting soluble amyloid β precursor protein 770β derived from vascular endothelial cell, comprising the following steps:
(1) a step of contacting a biological sample derived from a test subject suspected of having a disease accompanied by accumulation of amyloid β peptide with an antibody recognizing soluble amyloid β precursor protein β or an amyloid β precursor protein 770-specific antibody; and
(2) a step of detecting soluble amyloid β precursor protein 770β in the complex formed in step (1).

### TECHNICAL FIELD

The present invention relates to a method of detecting soluble amyloid β precursor protein 770β. More specifically, the present invention relates to a method of detecting an amyloid β precursor protein 770β cleavage product in a biological sample derived from a test subject suspected of having a disease accompanied by accumulation of amyloid β peptide, and a diagnostic reagent and a diagnosis kit for diseases accompanied by accumulation of amyloid β peptide, which are used for said method.

### BACKGROUND ART

With the recent advent of rapidly aging society in advanced countries, senile dementia is posing medically and socially serious problems. Measures against dementia such as Alzheimer's disease (hereinafter sometimes to be abbreviated as “AD”), particularly, establishment of an early diagnosis method, is urgently needed. Pathologically, AD is a disease confirmed to show senile plaque in the cerebral parenchyma mainly constituted with an aggregate of amyloid β peptide (hereinafter sometimes to be abbreviated as “Aβ”), vascular amyloid that appears due to Aβ accumulation in the cerebral blood vessels, intracellular accumulation of neurofibril tangle in the brain and the like (non-patent documents 1 and 2). Aβ is produced from an amyloid β precursor protein (hereinafter sometimes to be abbreviated as “APP”) by sequential enzyme reactions of β secretase (BACE1) (non-patent documents 3-6) and γ secretase (non-patent document 7). Since most of early-onset familial AD patients have a gene mutation influencing Aβ production mechanism or aggregation, and neurite associated with Aβ aggregation is often damaged (non-patent document 2), the process of Aβ aggregation and accumulation in the brain is considered to be strongly related to the onset of AD.

While APP is suggested to have a receptor-like function and binds to various extracellular matrix proteins (e.g., heparin and collagen (non-patent documents 8 and 9) etc.), elucidation of the biological function of APP still remains to be an important scientific problem (non-patent document 10). In mammals, two kinds of APP paralogs are known and designated as APP-like proteins 1 and 2 (APLP1 and APLP2, respectively). Interestingly, a triple knockout mouse that lacks all of these three APP family members (i.e., APP, APLP1 and APLP2) dies soon after birth (non-patent document 11), which suggests redundant function of APP family proteins. Furthermore, APP contains three kinds of isoforms APP695, APP751 and APP770 due to selective splicing (non-patent documents 12 and 13). As compared to APP695, APP751 further contains a Kunitz-type protease inhibitor (KPI) region, and APP770 contains an OX2 region in addition to KPI region. While APP695 is expressed in the highest amount in neuron, APP751 and APP770 show more universal expression patterns (non-patent document 14). Secretory APP containing the KPI region (also known as protease nexin 2) has a possibility of inhibiting a particular serine protease (particularly some of prothrombotic enzymes (non-patent document 15)). On the other hand, as for the function of the OX2 region, available information is extremely limited.

While many markers showing a correlation with AD have conventionally been reported, most of the markers are unclear as to the relationship with pathological changes, and diagnostic values have not been necessarily established. Examples of the marker directly related to the pathological changes include decrease of a kind of Aβ consisting of 42 amino acids (Aβ1-42), increase of (phosphorylated) tau protein and the like in the cerebrospinal fluid. However, Aβ is known to show value changes only after aggravation of the symptoms of Alzheimer's disease and cannot be used as an early diagnostic marker. When phosphorylated tau is used as a biomarker, the nerve cell death has already progressed at the time the phosphorylated tau shows an increase, and complete recovery of neural function cannot be expected by starting the treatment at this stage. In addition, since these markers are mainly measured in the cerebrospinal fluid (lumbar puncture fluid), sampling thereof requires a special technique, placing a great burden on patients, and cannot be a method for mass screening.

The symptoms of AD are reported to be closely related to Aβ accumulation in the cerebral parenchyma and intracerebral vascular wall (non-patent documents 16-18). Particularly, accumulation of Aβ in the cerebrovascular system is also considered to be the etiology of cerebrovascular amyloid angiopathy (hereinafter sometimes to be abbreviated as “CAA”). CAA is found in more than 80% of AD patients, and 10-40% of the elderly without AD (non-patent document 19). For example, according to an elaborated analysis of pathological section of patients with Alzheimer's disease by using electron microscope, amyloid fiber, which is Aβ aggregate, and the like are found in various intracerebral capillaries, which suggests that aggregation and accumulation of Aβ in the cerebral blood vessels are strongly related to AD (non-patent document 20). In addition, CAA is known to also cause cerebrovascular dementia disorder, cerebral apoplexy and intracerebral bleeding. In AD treatment using a vaccine against Aβ, moreover, CAA is suggested to be a potential influence causing side effects (non-patent documents 21 and 22), and therefore, the necessity of CAA diagnosis showing the state of Aβ accumulation in the cerebral blood vessels has widely been pointed out. While the important role of cerebrovascular smooth muscle cell in the removal of Aβ in blood vessels has recently attracted much attention (non-patent document 23), no precedent detailed analysis of APP in vascular endothelial cell is available, and the production site of accumulative vascular Aβ remains unrevealed.

### DOCUMENT LIST

### Non-Patent Documents

non-patent document 1: Physiol Rev 2001 81(2):741-766.
non-patent document 2: Cell 2005 120(4):545-555.
non-patent document 3: Nature 1999 402(6761):533-537.
non-patent document 4: Science 1999 286(5440):735-741.
non-patent document 5: Nature 1999 402(6761):537-540,
non-patent document 6: Nature 1999 398(6727):513-517.
non-patent document 7: Nature 1999 398(6727):518-522.
non-patent document 8: J Biol Chem 1996 271(3):1613-1620.
non-patent document 9: J Neurosci 1992 12(11):4143-4150.
non-patent document 10: Embo J 2005 24(23):3996-4006.
non-patent document 11: Embo J 2004 23(20):4106-4115.
non-patent document 12: Nature 1988 331(6156):525-527.
non-patent document 13: Nature 1988 331(6156):528-530.
non-patent document 14: Proc Natl Acad Sci USA 1993 90(20):9513-9517.
non-patent document 15: Proc Natl Acad Sci USA 2005 102(50):18135-18140.
non-patent document 16: Ann Neurol 1991 30(5):637-649.
non-patent document 17: Science 1990 248(4959):1120-1122.
non-patent document 18: Nat Genet 2006 38(1):24-26.
non-patent document 19: Stroke 2004 35(11 Suppl 1):2616-2619.
non-patent document 20: Ann N Y Acad Sci. 1997 Sep. 26; 826:25-34.
non-patent document 21: J. Neurosci. 2005 25, 6213-6220
non-patent document 22: J. Neurosci. 2005 25, 629-636
non-patent document 23: Nat Cell Biol 2009 11(2):143-153.

### SUMMARY OF THE INVENTION

### Problems to be Solved by the Invention

The search of a marker showing correlation with Alzheimer's disease and development of an early diagnosis method have conventionally been performed based on the production of Aβ in the nerve cell. However, a promising achievement for practical use has hardly been obtained.

The present invention aims to provide a method of detecting a biomarker, which enables an early diagnosis of a disease accompanied by accumulation of amyloid β peptide, such as Alzheimer's disease and the like.

### Means of Solving the Problems

In view of the above-mentioned problem, the present invention focuses the accumulation of Aβ in the cerebral blood vessel, which is the etiology of cerebrovascular amyloid angiopathy and the like, and is also correlated with the symptom of Alzheimer's disease. The present inventors have considered that Aβ production in the nerve cell and Aβ production in the vascular endothelial cell can be identified by separating nerve cell-derived APP and vascular endothelial cell-derived APP, and conducted intensive studies and analyzed.

As a result, they have found for the first time in the world that unique splicing variant APP770 can be specifically detected in vascular endothelial cell, which is conventionally unnoted from the aspect of Aβ production, and amyloid 1340 and 42 are produced from APP770 of vascular endothelial cell, and it leads to the finding that a cleavage product of APP770 can be used as an index of amyloid accumulation in the cerebral blood vessels.

The present invention enables diagnosis of the onset of a disease accompanied by Aβ accumulation, or prediction of the risk of the onset thereof, such as cerebrovascular amyloid angiopathy caused by Aβ accumulation in the cerebral blood vessel, and the like, or Alzheimer's disease correlated with the accumulation of Aβ in the cerebral blood vessel and the like, by specifically detecting a cleavage product of APP770. The present inventors have found that soluble APP770β (sAPP770β), which is an N-terminal side fragment of BACE1 cleavage product of APP770, can be a biomarker of diseases accompanied by Aβ accumulation, such as cerebrovascular amyloid angiopathy and Alzheimer's disease, since Aβ and C-terminal side fragment of APP after Aβ production are common in each APP splicing variant, which resulted in the completion of the present invention.

More specifically, the present invention provides the following.

[1] A method of detecting soluble amyloid β precursor protein 770β derived from vascular endothelial cell, comprising the following steps:
(1) a step of contacting a biological sample derived from a test subject with an antibody recognizing soluble amyloid β precursor protein β or an amyloid β precursor protein 770-specific antibody; and

(2) a step of detecting soluble amyloid β precursor protein 770β in the complex formed in step (1).

[2] The detection method of [1], wherein, in step (1), one of the antibody recognizing soluble amyloid β precursor protein β and the amyloid β precursor protein 770-specific antibody is used, and
step (2) comprises
(2a) a step of contacting soluble amyloid β precursor protein 770β in the complex formed in step (1) with the antibody recognizing soluble amyloid β precursor protein β or amyloid β precursor protein 770-specific antibody, which was not used in step (1); and (2b) a step of detecting the complex formed in step (2a).
[3] The method of [1] or [2], wherein the biological sample is blood, plasma or serum.
[4] The method of [1] or [2], wherein the biological sample is cerebrospinal fluid.
[5] The method of [1] or [2], wherein the biological sample is culture supernatant of vascular endothelial cell.
[6] The method of any one of [1] to [5], comprising, in addition to steps (1) and (2), detection of soluble amyloid β precursor protein 695β by the following steps:
(3) a step of contacting a biological sample derived from the test subject suspected of having a disease accompanied by accumulation of amyloid β peptide, with an antibody recognizing soluble amyloid β precursor protein β or soluble amyloid β precursor protein 695β-specific antibody; and
(4) a step of detecting soluble amyloid β precursor protein 695β in the complex formed in step (3).
[7] The method of [6], wherein, in step (3), one of the antibody recognizing soluble amyloid β precursor protein β and the amyloid β precursor protein 695-specific antibody is used, and
step (4) comprises
(4a) a step of contacting soluble amyloid β precursor protein 695β in the complex formed in step (3), with the antibody recognizing soluble amyloid β precursor protein β or soluble amyloid β precursor protein 695β-specific antibody, which was not used in step (3); and
(4b) a step of detecting the complex formed in step (4a).
[8] The method of [6] or [7], wherein the biological sample in step (1) is blood, plasma, serum, culture supernatant of vascular endothelial cell or cerebrospinal fluid, and the biological sample in step (3) is cerebrospinal fluid.
[9] The method of any one of [1] to [8], wherein the amyloid β precursor protein 770-specific antibody is an antibody recognizing OX2 domain.
[10] The method of any one of [1] to [9], wherein the test subject is suspected of having a disease accompanied by accumulation of amyloid β peptide.
[11] The method of [10], wherein the disease is at least any one of cerebrovascular amyloid angiopathy, cerebrovascular dementia, cerebral infarction and Alzheimer's disease.
[12] The method of any one of [1] to [9], wherein the test subject is suspected of having Alzheimer's disease occurring in association with at least any one of cerebrovascular amyloid angiopathy, cerebrovascular dementia and cerebral infarction.
[13] A diagnostic reagent for a disease accompanied by accumulation of amyloid β peptide, comprising an antibody recognizing soluble amyloid β precursor protein β and/or an amyloid β precursor protein 770-specific antibody as an active ingredient.
[14] A kit for diagnosis of a disease accompanied by accumulation of amyloid β peptide, comprising an antibody recognizing soluble amyloid β precursor protein β and an amyloid β precursor protein 770-specific antibody.
[15] The kit of [14], further comprising a soluble amyloid β precursor protein 695β-specific antibody.

[16] The diagnostic reagent of [13], or the kit of [14] or [15], wherein the target disease is at least any one of cerebrovascular amyloid angiopathy, cerebrovascular dementia, cerebral infarction and Alzheimer's disease.

[17] The diagnostic reagent or kit of [16], wherein the target disease is concurrence of at least any one of cerebrovascular amyloid angiopathy, cerebrovascular dementia and cerebral infarction, and Alzheimer's disease.
[18] A method of determining the possibility of having a disease accompanied by accumulation of amyloid β peptide by detection of soluble amyloid β precursor protein 770β derived from vascular endothelial cell, comprising the following steps:
(1) a step of contacting a biological sample obtained from a test subject, with an antibody recognizing soluble amyloid β precursor protein β or amyloid β precursor protein 770-specific antibody; and
(2) a step of detecting soluble amyloid β precursor protein 770β in the complex formed in step (1).
[19] The detection method of [18], wherein, in step (1), one of the antibody recognizing soluble amyloid β precursor protein β and the amyloid β precursor protein 770-specific antibody is used, and
step (2) comprises
(2a) a step of contacting soluble amyloid β precursor protein 770β in the complex formed in step (1) with the antibody recognizing soluble amyloid β precursor protein β or amyloid β precursor protein 770-specific antibody, which was not used in step (1); and
(2b) a step of detecting the complex formed in step (2a).
[20] The method of [18] or [19], wherein the biological sample is blood, plasma or serum.
[21] The method of [18] or [19], wherein the biological sample is cerebrospinal fluid.
[22] The method of [18] or [19], wherein the biological sample is culture supernatant of vascular endothelial cell.
[23] The method of any one of [18] to [22], comprising, in addition to steps (1) and (2), detection of soluble amyloid β precursor protein 695β by the following steps:
(3) a step of contacting a biological sample obtained from the test subject, with an antibody recognizing soluble amyloid β precursor protein β or soluble amyloid β precursor protein 695β-specific antibody; and
(4) a step of detecting soluble amyloid β precursor protein 695β in the complex formed in step (3).
[24] The method of [23], wherein, in step (3), one of the antibody recognizing soluble amyloid β precursor protein β and the amyloid β precursor protein 695-specific antibody is used, and step (4) comprises
(4a) a step of contacting soluble amyloid β precursor protein 695β in the complex formed in step (3), with the antibody recognizing soluble amyloid β precursor protein β or soluble amyloid β precursor protein 695β-specific antibody, which was not used in step (3); and
(4b) a step of detecting the complex formed in step (4a).
[25] The method of [23] or [24], wherein the biological sample in step (1) is blood, plasma serum, culture supernatant of vascular endothelial cell or cerebrospinal fluid, and the biological sample in step (3) is cerebrospinal fluid.
[26] The method of any one of [18] to [25], wherein the amyloid β precursor protein 770-specific antibody is an antibody recognizing OX2 domain.
[27] The method of any one of [18] to [26], wherein the disease accompanied by accumulation of amyloid β peptide is at least any one of cerebrovascular amyloid angiopathy, cerebrovascular dementia, cerebral infarction and Alzheimer's disease.

[28] The method of any one of [18] to [26], for determining the possibility of having at least any one disease of cerebrovascular amyloid angiopathy, cerebrovascular dementia and cerebral infarction by detecting soluble amyloid β precursor protein 770β.

[29] A method of detecting soluble amyloid β precursor protein 770β derived from vascular endothelial cell, comprising the following steps:
(1′) a step of contacting a biological sample derived from a test subject suspected of having a disease accompanied by accumulation of amyloid β peptide with an amyloid β precursor protein 770-specific antibody or antibody recognizing soluble amyloid β precursor protein β; and
(2′) a step of detecting (preferably quantitatively detecting) soluble amyloid β precursor protein 770 in the complex formed in step (1′).

### Effect of the Invention

Since the detection method of the present invention uses sAPP770β produced in vascular endothelial cells as a measurement target, which is a biomarker of a disease accompanied by accumulation of amyloid β peptide, it enables convenient detection with high sensitivity using various body fluids such as blood, serum, plasma and cerebrospinal fluid. Moreover, since the method enables determination of Aβ production in nerve cell and Aβ production in vascular endothelial cell, it can diagnose the diseases such as cerebrovascular amyloid angiopathy and the like in which Aβ accumulation in the cerebral blood vessels is the etiology, and Alzheimer's disease in which Aβ accumulation in cerebral parenchymal region is the etiology as independent disease targets, or diagnose both diseases, suspecting concurrence. In addition, since sAPP770β is produced by cleavage by BACE1, which is the rate determining reaction for amyloid β peptide production, the amount of sAPP770β is considered to reflect the production amount of amyloid β peptide. In other words, utilizing the results obtained by the detection method of the present invention, whether or not the test subject is affected with the disease and whether or not the probability of developing the disease is high can be determined even in an early stage such as before onset or incipient stage. When blood, serum or plasma is used, the burden on the patients can be further reduced as compared to the use of the cerebrospinal fluid.

The kit for diagnosis and the diagnostic reagent of the present invention are suitable for the above-mentioned detection method of the present invention, and enable an efficient diagnosis of a disease accompanied by accumulation of amyloid β peptide.

### BRIEF DESCRIPTION OF THE DRAWINGS

FIG. 1 shows analysis of APP in vascular endothelial cell. FIG. 1A: schematic showing of 3 kinds of APP splice variants, APP695, APP751 and APP770, which shows a series of anti-APP antibodies, and the recognition sites of primers A, B, C and D used for the analysis of APP transcript. FIG. 1B: Western blot analysis of APP in cell lysates (20 μg protein) of human brain microvascular endothelial cell (BMEC), mouse primary neuron and human umbilical cord blood-derived vascular endothelial cell (HUVEC), using anti-APP C15, anti-KPI, anti-OX2, anti-PECAM and anti-GAPDH antibody. FIG. 10: detection of each APP transcript in BMEC, neuron, HUVEC and mouse liver sinusoidal endothelial cell (LSEC), wherein total RNA was extracted from each cell, reversely transcribed, then subjected to PCR analysis, and the amplification product was detected. A plasmid incorporating APP695, APP751 and APP770 was used as the standard.

FIG. 2 shows a series of lectin precipitates of endothelial APP. BMEC lysate (100 μg) was incubated with SSA-, MAA-, E4-PHA-, ConA-, RCA120- or Jacalin-agarose at 4° C. for 16 hr. The sample precipitated with lectin was washed and analyzed by Western blot using an anti-APP C15 antibody.

FIG. 3 shows analyses of APP695, APP751 and APP770 expressed in COS cells, wherein human APP695, APP751 and APP770 were independently overexpressed in COS cells, the obtained cell lysates (1 μg) were analyzed together with BMEC lysate (5 μg) and neuron lysate (10 μg) by Western blot using an anti-APP C15 antibody.

FIG. 4 shows glycosidase digestion of brain endothelial APP. FIG. 4A shows that high molecular weight type and low molecular weight type APP770 (APP-H and APP-L, respectively) have N-linked sugar chain. FIG. 4B shows that APP-H has sialylated core 1 type O-linked sugar chain. FIG. 4C is a schematic showing of APP770 variant used for the test. FIG. 4D shows change of mobility of wild-type APP770 and variant thereof expressed in COS cell on SDS-PAGE. The black and gray arrowheads show APP-H and APP-L, respectively.

FIG. 5 shows the analysis of sAPP secreted from BMEC. FIG. 5A: intact APP and soluble secretory sAPP pulled down from the cell lysate (C, 6 μg) and medium (M) of BMEC and mouse primary neuron by using heparin-agarose were analyzed by Western blot using an anti-APP 22C11 antibody. FIG. 5B: sAPP pulled down from BMEC by using heparin-agarose was incubated in the presence or absence of PNGase, sialidase and O-glycosidase, and analyzed by Western blot using an anti-APP 22C11 antibody. FIG. 5C: after cell surface biotinylation of BMEC, biotinylated cell surface protein was precipitated with streptavidin-sepharose, and analyzed by Western blot using an anti-APP C15 antibody. FIG. 5D: BMEC was cultured in the presence or absence of benzylGalNAc, and intact APP and sAPP in the cell lysate (C) and medium (M) were analyzed by Western blot using an anti-APP C15 antibody and an anti-APP 22C11 antibody, respectively.

FIG. 6 shows expression of APP770 in human cerebral blood vessel, cleavage at α and β sites, and secretion into CSF. FIG. 6A: sAPP770 secreted from BMEC was analyzed by Western blot using an anti-APP 22C11, anti-OX2, anti-sAPPα and anti-sAPPβ antibodies. FIG. 6B: Aβ40 and Aβ42 levels of the medium from BMEC cultured in Opti-MEM were analyzed (n=3) (upper panel). The ratio of Aβ42/Aβ40 secreted from BMEC and neuron is shown as mean±SEM (n=4) (lower panel). FIG. 6C: human brain section embedded in paraffin was analyzed by hematoxylin-eosin staining (upper panel a) and immunostaining using anti-OX2 antibody (lower panel b). The gray and black arrowheads in a show endothelial cell nucleus and smooth muscle cell nucleus, respectively. The arrow in b shows OX2 immunoreactive endothelium. Scale bar: 20 μm. FIG. 6D: sAPP in human CSF sample (each 0.5 ml) was pulled down with heparin-agarose (30 μl), and analyzed by Western blot using an anti-APP 22C11, anti-OX2 and anti-sAPPβ antibodies. The gray and black arrowheads show sAPPβ other than sAPP770β and sAPP770β, respectively.

### DESCRIPTION OF EMBODIMENTS

### Definition

In the present invention, amyloid β precursor protein (APP) means a molecule cleaved by β secretase (BACE1) which produces amyloid β peptide (in the present specification, to be referred to as Aβ peptide, or simply Aβ). According to the target to be detected, APP to be used is desirably derived from the same species as the target. As one example, 3 kinds of splicing variants of APP695, APP751 and APP770 are known for human APP.

The amino acid sequence of APP and a base sequence encoding same have been made public for several kinds of animals. As for human, the amino acid sequences (GenBank Accession Nos. NP_{—}958817, NP_{—}958816 and NP_{—}000475, respectively) and the base sequences (GenBank Accession Nos. NM_{—}201414.1, NM_{—}201413.1 and NM_{—}000484.2, respectively) of 3 kinds of APP splicing variants of APP695, APP751 and APP770 have been made public, and can be isolated by a method known per se. The information of amino acid sequence and base sequence of APP derived from an animal other than human can also be obtained easily by searching the genome databases of various animals, and can be isolated by a method known per se based on the information. The following explanation is based on human APP as an example.

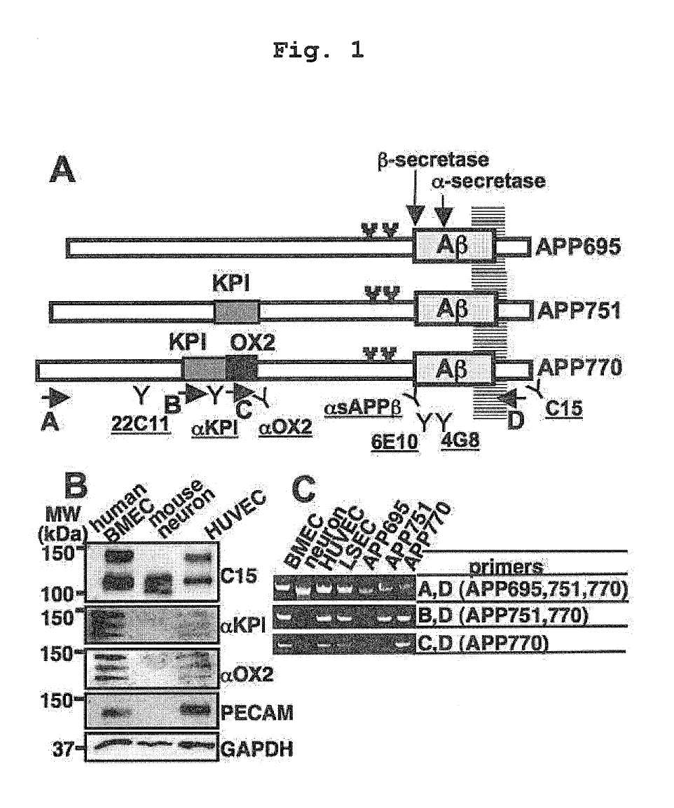

FIG. 1A shows the structure of APP splicing variant. As compared to APP695, APP751 further contains a Kunitz-type protease inhibitor (KPI) region, and APP770 contains OX2 region in addition to the KPI region. These 3 splicing variants have the same amino acid sequence in a region other than KPI region and OX2 region (including C-terminal side region containing Aβ region and transmembrane region). Therefore, the OX2 region is a characteristic sequence usable for distinguishing APP770 from the other two variants.

The amino acid sequence of human APP770 and the base sequence of nucleotide encoding the same are shown in SEQ ID NOs: 2 and 1, respectively. In the sequence of SEQ ID NO: 2, the KPI region corresponds to the 288-344th amino acid sequence, the OX2 region corresponds to the 345-364th amino acid sequence, and the Aβ40(42) region corresponds to the 672-711 (713)th amino acid sequence.

In the present invention, soluble amyloid β precursor protein β (sAPPβ) means an N-terminal side fragment of APP produced by cleavage with β secretase (called β cleavage), which does not include the C-terminal side fragment. The β cleavage products produced from APP splicing variants of APP695, APP751 and APP770 are referred to as sAPP695β, sAPP751β and sAPP770β, respectively. The amino acid sequence of human sAPP770β corresponds to the 18-671st amino acid sequence in the sequence of SEQ ID NO: 2.

Soluble amyloid β precursor proteinα (sAPPα) means an N-terminal side fragment of APP, which is produced by cleavage of APP with α secretase (called a cleavage) known as an Aβ non-productive pathway, and the α site means the cleavage site thereof.

The endothelial cell in the present invention refers to any vascular endothelial cell existing in the body, and particularly means vascular endothelial cells existing in any circulatory system from heart to capillary. The endothelial cell is specifically one-layer flat cells covering the inner wall of blood vessels. Preferable examples thereof include vascular endothelial cells such as brain microvascular endothelial cell, umbilical vein vascular endothelial cell, sinusoidal endothelial cell etc., and the like.

The antibody in the present invention comprises, but is not limited to, all classes and subclasses of immunoglobulin and also comprises forms of functional fragment of antibody, and the said antibody comprises natural antibodies and also comprises antibodies, antibody fragments, and binding fragments of these, which can be produced by gene recombination technique. The said antibody also comprises polyclonal antibodies which are antibody preparations containing different antibodies against different epitopes, and monoclonal antibodies which are antibodies obtained from substantially uniform antibody population (including antibody fragments). A binding fragment means a partial region of one of the above-described antibodies; specifically including, for example, F(ab′)₂, Fab′, Fab, Fv (variable fragment of antibody), sFv, dsFv (disulphide stabilized Fv), dAb (single domain antibody) and the like (Exp. Opin. Ther. Patents, Vol. 6, No. 5, p. 441-456, 1996).

In the present invention, the disease accompanied by accumulation of amyloid β peptide refers to a disease or condition suggested to involve a cause-effect relationship between the Aβ level in the body and the disease.

In a subject having a high Aβ level in the body, typically, accumulation of Aβ in the cerebral parenchymal region (called senile plaque) and accumulation thereof in the cerebral blood vessel (called cerebrovascular amyloid angiopathy (CAA)) are observed. Furthermore, destruction of nerve cells, cerebrovascular disorder and the like occur and, in a state where these have progressed, symptoms are exteriorized as dementia. As aging proceeds, the Aβ level in the body is considered to increase. Therefore, a high Aβ level in the body can occur even in a normal aging phenomenon; however, more rapid increase is observed in Alzheimer's disease. Thus, a disease accompanied by accumulation of amyloid β peptide typically includes, irrespective of whether the symptom is exteriorized, Alzheimer's disease, cerebrovascular amyloid angiopathy, cerebrovascular dementia, other dementia, cerebral infarction and the like. Since some reports indicate that Aβ accumulation in cerebral blood vessels is observed in initial stages of Alzheimer's disease, soluble amyloid β precursor protein 770β may increase before highly toxic Aβ oligomer increases.

### 1. Detection Method of Soluble Amyloid β Precursor Protein 770β

The detection method of the endothelial cell-derived soluble amyloid β precursor protein 770β of the present invention comprises the following steps:

(1) a step of contacting a biological sample derived from a test subject suspected of having a disease accompanied by accumulation of amyloid β peptide with an antibody recognizing soluble amyloid β precursor protein β or an amyloid β precursor protein 770-specific antibody; and
(2) a step of detecting (preferably quantitatively detecting) soluble amyloid β precursor protein 770β in the complex formed in step (1) (hereinafter to be also referred to as detection method I).
Step (1) is a step for contacting a biological sample derived from a test subject suspected of having a disease accompanied by accumulation of amyloid β peptide with an amyloid β precursor protein 770-specific antibody or antibody recognizing soluble amyloid β precursor protein β.

The test subject in step (1) is a subject to be determined as to whether it has a disease accompanied by accumulation of amyloid β peptide, and means a mammal suspected of having such disease. Examples of the mammal include experimental animals such as rodents (mouse, rat, hamster, guinea pig etc.), rabbit and the like, pets such as dog, cat and the like, domestic animals such as bovine, swine, goat, horse, sheep and the like, primates such as monkey, orangutan, chimpanzee and the like, human and the like, particularly preferably human. When rat or mouse is used, for example, a disease model such as chronic cerebral ischemia model and the like is generated, and changes of soluble amyloid β precursor protein 770β can be observed. Such test subject may or may not show cognitive impairment, and may be under treatment of a disease accompanied by accumulation of amyloid β peptide, such as Alzheimer's disease.

The biological sample can be recovered from the above-mentioned test subject by a known method. As the biological sample, various body fluids such as blood, plasma, serum, lymph fluid, cerebrospinal fluid and the like can be used.

In one embodiment, the biological sample of the present invention is blood, plasma or serum. When the method of the present invention is applied to mass screening, these samples are preferably used, since a special technique is not necessary for collection thereof, and a burden on the test subject is small.

In one embodiment, the biological sample of the present invention also includes cerebrospinal fluid. The cerebrospinal fluid fills and circulates in the brain tissue, and is considered to reflect brain states. It is also used to measure conventional biomarkers for brain states, and can be preferably used in the present invention as a biological sample.

In another embodiment, the biological sample of the present invention is a culture supernatant of vascular endothelial cells. The sensitivity in the detection method of the present invention can be enhanced by culturing and growing vascular endothelial cells. The vascular endothelial cells include brain microvascular endothelial cells, cord blood-derived vascular endothelial cells, liver sinusoidal endothelial cells, and the like. These cells may also be cells derived from stem cells, such as embryonic stem cells, tissue stem cells, and cells with pluripotency given by such as genetic engineering (e.g., iPS cells). The vascular endothelial cells can be collected from blood vessels surgically isolated from living organism, blood vessels for transplantation produced from ES cells and bone marrow cells, and the like. Alternatively, the vascular endothelial cells differentiated and grown from vascular endothelial progenitor cells present in blood (peripheral blood, cord blood, and the like) and adipose tissue can be used (see, for example, WO publication WO2006/090882 and JP-B-4217262). The vascular endothelial cells can be cultured by known methods and reagents.

A biological sample derived from a test subject is contacted with an amyloid β precursor protein 770 (APP770)-specific antibody or an antibody recognizing soluble amyloid β precursor protein β (sAPPβ) to form a complex of the soluble amyloid β precursor protein 770β and the antibody.

The “amyloid β precursor protein 770 (APP770)-specific antibody” in the present invention is an antibody having an ability to bind to APP770, which does not bind to a splicing variant of amyloid β precursor protein (APP) other than APP770 (APP695 and APP751 are known). The APP770-specific antibody also has an ability to bind to soluble amyloid β precursor protein 770β. While the antibody can be specific to APP770 derived from any mammal, it is preferably specific to APP770 derived from the same species as the test subject.

Such antibody can be prepared by a method known per se, based on the differences between APP770 and other splicing variants in the amino acid sequence, sugar chain addition state and the like. Specifically, for example, the APP770-specific antibody can be obtained by designing an antigen peptide sequence in the OX2 region present on the amino acid sequence of APP770 but absent on the amino acid sequences of APP695 and APP751, and producing an antibody using this peptide. Also, such antibody is commercially available (e.g., anti-OX2 antibody (Chemicon) etc.).

The term, “antibody recognizing soluble amyloid β precursor protein β (sAPPβ)” means antibody with ability to bind to all sAPPβs, including the above-mentioned sAPP695β, sAPP751β and sAPP770β, or at least sAPP770β. Such antibodies can be prepared by a method known per se, based on the information of amino acid sequence or the state of sugar chain addition and the like that are common in sAPPβs. Specifically, for example, an antibody which specifically recognizes sAPPβbut not sAPPα can be obtained by designing an antigen peptide sequence around the cleavage site by β secretase (β site) which is common in all APP variants, and then producing an antibody using said peptide. Alternatively, antibodies which can recognize sAPP751β and sAPP770β but not sAPP695β can be also obtained by designing an antigen peptide in the KPI region. Such antibodies are also commercially available (for example, mouse monoclonal anti-APP 22C11 antibody (Chemicon), anti-human sAPPβ antibody (IBL Co.) and anti-KPI antibody (Chemicon), and the like). Thus, in the present invention, although an antibody which further recognizes APPs without β cleavage (for example, anti-APP 22C11 antibody (Chemicon)) can be used as an “antibody recognizing soluble amyloid β precursor protein β”, from the aspects of specificity, an antibody recognizing the β site of soluble amyloid β precursor protein β (for example, anti-human sAPPβ antibody (IEL Co.)) is preferably used.

According to the present invention, moreover, an antibody specifically recognizing sAPP695β can be used for determining Aβ production in vascular endothelial cell in contrast with that in nerve cell and the invention includes, in addition to steps (1) and (2), the following steps:

(3) a step of contacting a biological sample derived from the test subject suspected of having a disease accompanied by accumulation of amyloid β peptide, with an antibody recognizing soluble amyloid β precursor protein β or soluble amyloid β precursor protein 695β-specific antibody; and
(4) a step of detecting soluble amyloid β precursor protein 695β in the complex formed in step (3), which enable detection of soluble amyloid β precursor protein 695β as well.

The term “sAPP695β specific antibody” means an antibody with ability to bind to the above-mentioned sAPP695β, but not to sAPP770β and sAPP751β. Such antibody can be prepared by a method known per se, based on the information of amino acid sequence or the state of sugar chain addition and the like of sAPP695β. Specifically, for example, an antibody specifically recognizing sAPP695β may be an antibody recognizing 40 amino acid sequence, preferably 30 amino acid sequence, more preferably from 10 to 20 amino acid sequence including the region in which the amino acid sequence from 288^{th} to 364^{th} amino acid (KPI region and OX2 region are present in this sequence) is removed, and 287^{th} and 365^{th} amino acids are linked in SEQ ID NO: 2. An antibody specifically recognizing sAPP695β but not sAPP770β and sAPP751β can be obtained by designing antigen peptides having these sequences and producing antibodies with said peptides. Such antibodies have an ability to specifically bind to APP695, but not to APP770 and APP751.

Also, in the step (3), an antibody specifically recognizing sAPP751β can be appropriately used. The term, “sAPP751β specific antibody” means an antibody with ability to bind to the above-mentioned sAPP751β, but not to sAPP770β and sAPP695β. Such antibody can be prepared by a method known per se, based on the information of amino acid sequence or the state of sugar chain addition and the like of sAPP751β. Specifically, for example, an antibody specifically recognizing sAPP751β may be an antibody recognizing 40 amino acid sequence, preferably 30 amino acid sequence, more preferably from 10 to 20 amino acid sequence including the region in which the amino acid sequence from 345^{th} to 364^{th} amino acid in the OX2 region is removed and the potion the KPI region resides, i.e. the potion 344^{th} and 365^{th} amino acids are linked, in SEQ ID NO: 2. An antibody specifically recognizing sAPP751β but not sAPP770β and sAPP695β can be obtained by designing antigen peptides of these sequences and producing antibodies with said peptides. Such antibodies have an ability to specifically bind to APP751, but not to APP770 and APP695.

The APP770-specific antibody, sAPP695β-specific antibody, sAPP751β-specific antibody and antibody recognizing sAPPβmay be either a polyclonal antibody or a monoclonal antibody. Said antibodies also include, but are not limited to, besides natural antibodies, antibodies that can be produced by gene recombination techniques, antibody fragments and binding fragments thereof. The binding fragment means a part of the region of the aforementioned antibody. Specific examples thereof include F(ab′)₂, Fab′, Fab, Fv (variable fragment of antibody), sFv, dsFv (disulphide stabilized Fv), dAb (single domain antibody) and the like (Exp. Opin. Ther. Patents, Vol. 6, No. 5, p. 441-456, 1996).

The aforementioned antibodies may be directly or indirectly labeled with a labeling substance. Examples of the labeling substance include fluorescent substances (e.g., FITC, rhodamine), radioactive substances (e.g., ³²P, ³⁵S, ¹⁴C, ³H), enzymes (e.g., alkaline phosphatase, peroxidase), colored particles (e.g., colloidal metal particles, colored latex), biotin and the like.

The aforementioned antibody can be also used in the soluble state without conjugation, or may be conjugated to solid phase. The “solid phase” includes a plate (e.g., microwell plate), a tube, beads (e.g., plastic beads, magnetic beads), a carrier for chromatography (e.g., water-absorbing substrate such as nitrocellulose membrane, Sepharose), a membrane (e.g., nitrocellulose membrane, PVDF membrane), gel (e.g., polyacrylamide gel), metal film (e.g., gold film), and the like. Of these, a plate, beads, a carrier for chromatography, and a membrane are preferably used, and a plate is most preferably used due to ease of handling. The above-mentioned conjugation includes, but is not particularly limited to, covalent bond, ionic bond, physical adsorption, and the like, and covalent bond and/or physical adsorption are preferable, due to their sufficient bond strength. The conjugation to solid phase may be direct conjugation to solid phase, or may be indirect conjugation to solid phase utilizing substances known per se.

In addition, it is a general practice to contact a solid phase with a phosphate-buffered solution of bovine serum albumin (BSA), cow milk protein and the like to suppress non-specific adsorption and non-specific reaction, by blocking, with the aforementioned BSA, cow milk protein etc., and the like, a solid phase surface moiety not coated with an antibody.

The embodiment, order, specific method, and the like to contact APP770-specific antibody, sAPP695β-specific antibody, sAPP751β-specific antibody, or antibody recognizing sAPPβ with biological sample derived from test subject suspected to have a disease with Aβ accumulation are not particularly limited if it is a method that these antibodies are able to interact with sAPP770β etc. in the biological sample. The contact may be performed, for example, by adding biological sample to a plate in which APP770-specific antibodies or antibodies recognizing sAPPβ are solid phased. Alternatively, for example, it may be also performed by contacting antibodies with biological sample separated by a method such as SDS-PAGE, and transferred and immobilized on a membrane.

While the time of the contact to be maintained is not particularly limited as long as it is sufficient for the aforementioned antibody and sAPP770β etc. contained in a biological sample derived from a test subject suspected of having a disease accompanied by Aβ accumulation to be bound to each other to form a complex, it is generally several seconds—over 10 hours. The temperature conditions for the contact is generally 4° C.-50° C., preferably 4° C.-37° C., most preferably room temperature of about 15° C.-30° C. The pH condition of the reaction is preferably 5.0-9.0, particularly preferably a neutral range of 6.0-8.0.

Step (2) is a step for detecting soluble amyloid β precursor protein 770β in the complex formed in the above-mentioned step (1).

For the above-mentioned detection, any method capable of detecting soluble amyloid β precursor protein 770β can be used. For example, a method for detection of sAPP770β based on the molecular weight thereof, or a detection method utilizing an antibody is used.

According to the present invention, moreover, a step of detecting sAPP695β can also be included with the aim to determine Aβ production in vascular endothelial cell in contrast with that in nerve cell. In this case, the detection of sAPP770β can be used as an index of Aβ production in vascular endothelial cell and the detection of sAPP695β can be used as an index of Aβ production in nerve cell. For the detection, any method capable of detecting sAPP695β can be used and, for example, a method for detection of sAPP695β based on the molecular weight thereof, or a detection method utilizing an antibody is used.

In addition, step (4) may include a step of detecting sAPP751β as appropriate as a further control. For the detection, any method capable of detecting sAPP751β can be used and, for example, a method for detection of sAPP751β based on the molecular weight thereof, or a detection method utilizing an antibody is used.

The detection of sAPP770β, sAPP695β or sAPP751β based on their molecular weight can be performed, but is not limited to, by detecting a band, a spot, or a peak at intended molecular weight, comparing with molecular weight of predefined marker peptides, after applying to measurement such as by gel electrophoresis (e.g., SDS-PAGE, and the like), various separation and purification methods (e.g., size-exclusion chromatography, ion exchange chromatography, hydrophobic chromatography, affinity chromatography, reversed-phase chromatography, and the like), and mass spectrometer (e.g., double-focusing mass spectrometer, quadrupole analyzer, time-of-flight mass spectrometer (TOF MS), Fourier-transform mass spectrometer, ion cyclotron mass spectrometer, and the like), and combination thereof.

Before detection of sAPP770β (or sAPP695β or sAPP751β), the above-mentioned complex may be isolated from the reaction mixture of step (1) (or (3)). Such isolation can be performed by, for example, an isolation method utilizing affinity for heparin, an immunoprecipitation method and the like, and a suitable label (for example, biotin and the like) added to the antibody used in step (1) (or (3)) may be utilized.

The above-mentioned detection may be performed after formation of a complex. Alternatively, it may be performed after liberation of sAPP770β, sAPP695β or sAPP751β by isolating the formed complex from the sample and dissociating the complex.

The measurement using a mass spectrometer is performed, for example, as follows. Firstly, the complex formed in step (1) or (3) is isolated by a method utilizing the label added to the antibody used in step (1) or (3) (e.g., immunoprecipitation method etc.). The isolated complex may be fragmentated by digesting with a protease such as trypsin and the like. Digestion with a protease having high sequence specificity enables identification of protein based on the pattern of the generated peptide. Then the sample is subjected to ionization and mass spectrometry. For ionization, an appropriate ionization method (e.g., electron impact ionization method, field desorption method, secondary ionization method, high-speed atom bombardment method, matrix assisted laser desorption/ionization (MALDI) method, electrospray ionization (ESI) method etc.) can be adopted, with preference given to MALDI method and ESI method, since they show properties superior in ionization of macromolecular compounds. Combination of the ionization method and mass spectrometry includes, for example, MALDI-TOF MS, ESI-MS and the like. In addition, tandem mass spectrometry (MS/MS) may also be combined. The obtained mass spectrum is compared with that of sAPP770β, sAPP695β or sAPP751β to detect sAPP770β, sAPP695β or sAPP751β.

When sAPP770β is detected in step (2), the test subject can be determined to be affected with a disease accompanied by accumulation of Aβ or have high probability of developing the disease. When sAPP770β is not detected or below detection limit, the test subject can be determined to be not affected with a disease accompanied by accumulation of Aβ or have low probability of developing the disease. While the disease is preferably cerebrovascular amyloid angiopathy, cerebrovascular dementia, or cerebral infarction, it may be Alzheimer's disease, and is not limited to these diseases.

Moreover, when sAPP695β, further, sAPP751β, was detected with the aim to contrast with Aβ production in nerve cell, the test subject can be determined to be affected with a disease accompanied by accumulation of Aβ in nerve cell, preferably Alzheimer's disease, or have high probability of developing the disease. When the target disease in the detection of sAPP770β is Alzheimer's disease, the aforementioned determination can determine the onset state of Alzheimer's disease in more detail.

When a method for detection of sAPP770β utilizing an antibody is adopted, the step (2) of the present invention preferably includes

(2a) a step of contacting soluble amyloid β precursor protein 770β in the complex formed in step (1) with the antibody recognizing soluble amyloid β precursor protein β or amyloid β precursor protein 770-specific antibody, which was not used in step (1); and
(2b) a step of detecting the complex formed in step (2a).

In addition, when a method for detection of sAPP695β utilizing an antibody is adopted, the aforementioned step (4) preferably includes

(4a) a step of contacting soluble amyloid β precursor protein 695β in the complex formed in step (3), with the antibody recognizing soluble amyloid β precursor protein β or soluble amyloid β precursor protein 695β-specific antibody, which was not used in step (3); and
(4b) a step of detecting the complex formed in step (4a).

The complex formed in step (1) can be directly contacted with an antibody, or after dissociation of the complex to liberate sAPP770β.

The explanation of the labeling of antibody, and contact method and contact conditions with sAPP770β can be the corresponding descriptions of the labeling of antibody, and contact method and contact conditions with a biological sample in the above-mentioned step (1).

Step (2b) is a step for detecting a complex formed by the complex formed in step (1) and the antibody used in step (2a), or a complex formed by sAPP770β and the antibody used in step (2a).

The detection can be performed by detecting sAPP770β contained in the complex or the antibody used in step (2a). Such detection can be performed for the complex as it is, or after dissociation of the complex to liberate soluble amyloid β precursor protein 770β.

The above-mentioned detection can be performed by enzyme immunoassay (EIA method), fluorescent immunoassay (FIA), immunochromatography method, Western blotting, radioimmunoassay and the like, and these analysis methods are well known to those of ordinary skill in the art.

When EIA method is selected as the detection method of step (2b), the detection method of the present invention is preferably performed by a sandwich ELISA method using an APP770 specific antibody and an antibody recognizing soluble amyloid β precursor protein 770β (sAPP770β). Such sandwich ELISA method is suitable for practicing the present invention since it has high specificity for antigen.

The sandwich ELISA method is performed, for example, as follows. Firstly, the antibody in step (1) is solid phased on the well surface of an ELISA plate. After blocking to prevent non-specific adsorption onto the well surface, a sample is added to allow contact of sAPP770β in the sample with the antibody to form a complex. Protein not bound with the antibody is removed by washing, the labeled antibody in step (2a) is added to the well to allow contact with sAPP770β to form a complex, and detection and quantification are performed using the label.

For sandwich ELISA method, an enzyme-labeled antibody can be used. As the enzyme usable as a label, peroxidase, alkaline phosphatase, glucose oxidase, β-galactosidase and the like can be exemplified. As the substrate used for the enzyme detection, a substrate suitable for the selected enzyme label is employed. For example, when peroxidase is selected as the enzyme, o-phenylenediamine (OPD), tetramethylbenzidine (TMB) and the like are used and, when alkaliphosphatase is selected, p-nitrophenylphosphate (PNPP) and the like are used. As the reaction stop solution and substrate solution, conventionally-known ones can be appropriately used without particular limitation, according to the selected enzyme.

As one kind of the sandwich ELISA method, a method utilizing an avidin-biotin reaction is applicable. In this method, for example, sAPP770β in the sample is trapped by the solid phased antibody used in step (1), and an antigen antibody reaction is performed between the trapped sAPP770β and the biotin-labeled antibody used in step (2a). Then, enzyme-labeled streptavidin is added to perform an avidin-biotin reaction. Then, sAPP770β is detected by detecting this enzyme.

The above-mentioned biotin-labeled antibody used in step (2a) can be produced by conjugating biotin to the antibody used in step (2a) by a method known per se. Such labeling can be performed using, for example, a commercially available biotin labeling kit. The enzyme-labeled streptavidin may also be a commercially available product.

In addition to the above-mentioned method, a method utilizing the secondary antibody is also applicable. In this method, the antibody used in step (1) and the antibody used in step (2a) are respectively derived from different animal species, and a complex is formed between a secondary antibody recognizing the Ig domain of the antibody used in step (2a) and the antibody used in step (2a). The secondary antibody used is labeled with an enzyme, and the presence of sAPP770β in the complex can be determined by detecting the enzyme. As the enzyme-labeled secondary antibody, a commercially available product can be used. Such method using the secondary antibody can enhance sensitivity of the detection method of the present invention.

When the FIA method is selected as a detection method of step (2b), sAPP770β can be detected according to a method similar to the above-mentioned sandwich ELISA method by substituting the enzyme used as a label in the above-mentioned EIA method with a fluorescent substance.

As the fluorescent substance, chemical substances such as APC, PE, Cy2, Cy3, Cy5, ECD, FITC, PerCP, Alexa (registered trade mark) Fluor, fluorescein, rhodamine and the like can be preferably utilized. Labeling with these chemical substances can be performed according to a method known per se.

Fluorescence can be detected using a commercially available measurement device, fluorescence microscope and the like.

When an immunochromatography method is selected as a detection method of step (2b), for example, a complex is formed among the antibody of step (2a), which is solid phased in a line on a water-absorbing substrate such as nitrocellulose membrane and the like, a biological sample developed on the membrane, and the labeled antibody of step (1). The labeled antibody of step (1) may be mixed in advance with the biological sample, or supplied on the water-absorbing substrate so that it will contact the biological sample before the biological sample contacts the antibody used in step (2a). The formation of complex can be detected by a method according to the label. For example, when gold colloid particles are used as a label, detection is possible since the region with accumulation of the labeled antibody turns red.

Western blotting can be chosen as a detection method of step (2b). The Western blotting (also called as immunoblotting) is a method to detect a particular protein in a sample. Gel electrophoresis is performed to separate proteins based on the length of polypeptides under a denaturing condition or based on three-dimensional structure of proteins under a non-denaturing condition. Subsequently, after proteins are transferred to and immobilized on a membrane (typically, nitrocellulose or PVDF), target proteins are detected using specific antibodies.

Furthermore, when determination of Aβ production in vascular endothelial cell in contrast with that in nerve cell is also desired, sAPP695β can be detected in steps (4a) and (4b) in the same manner as in the detection of sAPP770β in steps (2a) and (2b). When detection of sAPP751β is added as a further control, it can be performed in the same manner as in the detection of sAPP770β in steps (2a) and (2b).

The detection step in the present invention is preferably a step for quantitatively detecting sAPP770β. Quantification can be performed by a method known per se, such as digitizing the signal of the biological sample detected based on the label used, by utilizing the analytical curve formed using a standard sample of sAPP770β, and the like. The standard sample of sAPP770β may be sAPP770β prepared from a biological sample, or can also be prepared based on the information of the gene encoding sAPP770β, by a genetic engineering technique known per se.

When detection of sAPP695β is added as a detection target for determination of Aβ production in vascular endothelial cell in contrast with that in nerve cell, sAPP695β can be handled in the same manner as with sAPP770β in the aforementioned steps (2a) and (2b). The same applies when sAPP751β is detected.

Utilizing the results obtained by the method of the present invention, diagnosis can be made as to whether or not affected with a disease accompanied by accumulation of Aβ, and the possibility of developing the disease. Such diagnosis can be performed by comparing the level of sAPP770β in the biological sample derived from the test subject with the concentration range of sAPP770β in the control sample derived from healthy or young subjects (which can be examined by statistical study). The diagnosis can also be made by comparing with the level measured before for the same test subject.

The sAPP770β level to be the standard is an appropriate standard set according to the object. sAPP770β can increase in amount along with aging even in healthy individuals. Thus, “mean+2 standard deviation” of healthy individual in the same age range as the disease control group is set as a cut off value, and when the level exceeds the cut off value, a high possibility of “a disease accompanied by accumulation of amyloid β peptide” is diagnosed.

Also, the treatment effect for patients under treatment of diseases accompanied by accumulation of Aβ can also be evaluated using the method of the present invention. The effect of the treatment can be known by collecting biological samples from such patients before treatment, during treatment and/or after treatment, and examining changes of the concentration of sAPP770β. For example, when the concentration of sAPP770β in the later biological sample is lower than that of the earlier biological sample, the treatment can be evaluated as effective. Some therapeutic drugs for dementia are known to possibly cause CAA, and a risk prediction diagnosis of CAA in patients under administration of therapeutic drugs can also be performed by the method of the present invention.

According to the present invention, moreover, determination of Aβ production in vascular endothelial cell in contrast with that in nerve cell is possible. In this case, Aβ production in vascular endothelial cell can be determined using the level of sAPP770β in the biological sample as an index, and Aβ production in nerve cell can be determined using the level of sAPP695β in the biological sample as an index.

The sAPP770β level to be the index of Aβ production in vascular endothelial cell is utilized for judging whether being affected with a disease accompanied by accumulation of Aβ, preferably cerebrovascular amyloid angiopathy, cerebrovascular dementia, cerebral infarction or Alzheimer's disease, more preferably cerebrovascular amyloid angiopathy, cerebrovascular dementia or cerebral infarction, wherein Aβ accumulation in cerebral blood vessel is the etiology, or high probability of developing the disease. The sAPP695β level to be the index of Aβ production in nerve cell is utilized for judging whether being affected with a disease accompanied by accumulation of Aβ in cerebral parenchymal region, preferably Alzheimer's disease, or high probability of developing the disease.

In a diagnosis combining these determinations, using cerebrovascular amyloid angiopathy, cerebrovascular dementia or cerebral infarction and Alzheimer's disease as a target disease, or concurrence of these diseases as a target, these diseases can be determined as independent targets, or concurrent state of these diseases can be determined as a target, or the possibility thereof can be determined. In addition, when the target disease of the determination by the aforementioned sAPP770β level and that by the sAPP695β level are both Alzheimer's disease, the diagnosis can determine, in more detail, the state of Alzheimer's disease after the onset.

In addition, the present invention can be used in combination with known diagnosis of Alzheimer's disease, including but not limited to, as diagnoses of Alzheimer's disease other than the present invention, clinical diagnosis using NINCDS-ADRDA or DSM-IV as a diagnostic criteria, diagnosis method utilizing decrease of amyloid β peptide 42 in the body fluid as an index, diagnosis method utilizing increase of tau or phosphorylated tau in the body fluid as an index, diagnosis method utilizing increase or decrease of sugar chain metabolites by BACE1 activity in the body fluid as an index, imaging diagnosis by amyloid imaging utilizing aggregation of Aβ as an index, imaging diagnosis utilizing partial atrophy in the brain as an index, and the like.

Furthermore, when combined with the above-mentioned diagnosis of Alzheimer's disease, the present invention can also predict the risk of the side effect of increased microbleeding due to a certain kind of antibody in cerebrovascular amyloid angiopathy, which was reported in the immunotherapy of Alzheimer's disease using an antibody to amyloid β peptide (Hartman, R. E., et al., J. Neurosci., 25, 6213-6220 (2005), Racke, M. M., et al., J. Neurosci., 25, 629-636 (2005).).

Since it is possible that Aβ accumulation in vascular endothelial cells affects a symptom of the Alzheimer's disease by a transition to the cerebral parenchymal region, the detection of sAPP770β according to the present invention can be used as a diagnostic marker related to the pathology of Alzheimer's disease. This is based on the finding that emboli are leaked to outside of blood vessel in a recanalization of brain microvessel (Nature. 2010 May 27; 465(7297):478-82.). From this finding, since Aβ accumulation is also present as an embolus, it is possible that Aβ is leaked to outside of blood vessel in a recanalization of brain microvessel, and said Aβ consequently accumulate in the cerebral parenchymal region and affect the pathology of Alzheimer's disease.

Therefore, the detection of sAPP770β in the present invention enables diagnosis of Alzheimer's disease based on the possibility of accumulation of Aβ transferred from brain microvessel, or correlation with cerebrovascular amyloid angiopathy. The diagnosis of Alzheimer's disease based on the detection of sAPP695β takes note of the accumulation of Aβ produced from the nerve cell in the cerebral parenchymal region. Therefore, the diagnosis of Alzheimer's disease by the detection of sAPP770β enables diagnosis of Alzheimer's disease by a principle different from the diagnosis by the detection of sAPP695β. This also means that diagnosis of Alzheimer's disease based on the combination of the detection of sAPP770β and the detection of sAPP695β can provide a more detailed diagnosis of the disease state of Alzheimer's disease than the individual diagnosis.

In one embodiment, the detection method of soluble amyloid β precursor protein 770β of the present invention may be a detection method containing the following steps instead of steps (1) and (2)

(1′) a step of contacting a biological sample derived from a test subject suspected of having a disease accompanied by accumulation of amyloid β peptide with an amyloid β precursor protein 770-specific antibody or antibody recognizing soluble amyloid β precursor protein β; and
(2′) a step of detecting (preferably quantitatively detecting) soluble amyloid β precursor protein 770 in the complex formed in step (1′)
(hereinafter to be also referred to as detection method II).

Step (1′) is the same as step (1) of detection method I.

In step (2′), soluble amyloid β precursor protein 770 means an amyloid β precursor protein 770 cleavage product (N-terminal side fragment of APP770) containing at least sAPP770β. That is, in step (2′), other amyloid β precursor protein 770 cleavage product (N-terminal side fragment of APP770) (e.g., sAPP770a) can also be detected together with sAPP770β. When the test subject shows higher β cleavage activity than healthy human, the disease of the test subject can be substantially diagnosed, or the risk thereof can be predicted, by detecting (preferably quantitatively detecting) soluble amyloid β precursor protein 770 in step (2′).

As for other aspects, the description of detection method I can be applied to detection method II.

### 2. Diagnostic Reagent for Disease Accompanied by Accumulation of Amyloid β Peptide

The present invention provides a diagnostic reagent for a disease accompanied by accumulation of amyloid β peptide, which contains an amyloid β precursor protein 770-specific antibody and/or an antibody recognizing soluble amyloid β precursor protein β as an active ingredient. The antibody(antibodies) to be the active ingredient of the diagnostic reagent of the present invention is(are) the antibody recognizing soluble amyloid β precursor protein β and/or the amyloid β precursor protein 770-specific antibody described in “1. Detection method of soluble amyloid β precursor protein 770β”.

The diagnostic reagent of the present invention may consist only of the above-mentioned antibody(antibodies) alone or may contain a pharmaceutically acceptable carrier. When the diagnostic reagent of the present invention is prepared as a liquid, the pharmaceutically acceptable carrier may contain various carriers conventionally used as preparation materials, for example, diluent, solvent, solubilizing agent, suspending agent, isotonicity agent, buffering agent and the like. Moreover, to prevent aggregation of antibody, a surfactant such as Tween 20 (registered trade mark) and the like are preferably added. The mixing ratio thereof can be appropriately determined by those of ordinary skill in the art.

Using the diagnostic reagent of the present invention, whether a test subject is affected with a disease accompanied by accumulation of Aβ can be diagnosed by detecting sAPP770β derived from endothelial cell for a biological sample derived from the test subject suspected of having a disease accompanied by accumulation of amyloid β peptide, by the aforementioned detection method of the present invention. Using the diagnostic reagent of the present invention, the diagnosis of a disease accompanied by accumulation of Aβ becomes convenient and highly accurate. In one embodiment, the diagnostic reagent of the present invention for a disease accompanied by accumulation of Aβ is a diagnostic reagent for cerebrovascular amyloid angiopathy, cerebrovascular dementia, cerebral infarction or Alzheimer's disease.

### 3. Kit for Diagnosis of Disease Accompanied by Accumulation of Amyloid β Peptide

The present invention provides a kit for diagnosis of a disease accompanied by accumulation of amyloid β peptide, which contains an amyloid β precursor protein 770-specific antibody and an antibody recognizing soluble amyloid β precursor protein β as an active ingredient. The antibodies to be the active ingredient of the kit of the present invention are the antibodies described in “1. Detection method of soluble amyloid β precursor protein 770β”.

The diagnosis kit of the present invention may contain, besides the above-mentioned antibodies, antibody such as sAPP695β specific antibody, sAPP751β specific antibody and the like, or a reagent and the like. These antibody or reagent or the like may be combined with the above-mentioned antibodies in advance, or may be contained in separate containers. Examples of the antibody or reagent or the like include the secondary antibody, substrate, labeling substance (e.g., fluorescence dye, enzyme), solid phase, reaction container described in “1. Detection method of soluble amyloid β precursor protein 770β”, and treatment liquid, buffer for diluting antibody, positive control (e.g., recombinant sAPP770β), negative control, instruction describing protocol and the like. These factors can also be mixed in advance where necessary.

In the diagnosis kit of the present invention, the antibody may be solid phased in advance, and may be labeled in advance. The solid phase usable for the diagnosis kit of the present invention is not particularly limited and, for example, polymers such as polystyrene and the like, and insoluble carriers such as glass beads, magnetic particles, microplate, immunochromatography filter paper, glass filter and the like. Preferred is a microplate used for the sandwich ELISA method.

While the form of the diagnosis kit of the present invention is not particularly limited, an integrated diagnosis kit, wherein the components of the diagnosis kit of the present invention are integrated, can be provided to achieve convenient diagnosis. Examples of the form of the integrated diagnosis kit include a cassette type using an immunochromatography method.

Using the diagnosis kit of the present invention, the diagnosis of a disease accompanied by accumulation of Aβbecomes convenient and highly accurate. In one embodiment, the diagnosis kit of the present invention for a disease accompanied by accumulation of Aβ is used as a kit for diagnosis of cerebrovascular amyloid angiopathy, cerebrovascular dementia, cerebral infarction or Alzheimer's disease.

### EXAMPLES

The present invention is explained in detail in the following by referring to the following Examples, which are not to be construed as limitative. The reagent, apparatus and materials used in the present invention are commercially available unless otherwise indicated.

### (Materials)

The sources of materials used in this study are as follows: tissue culture medium and reagent (including DMEM) from Invitrogen; recombinant peptide N-glycosidase F (PNGase) from New England BioLabs; O-glycosidase from Roche; Arthrobacter ureafaciens sialidase from Nacalai Tesque; protein A-sepharose Fast Flow from GE Healthcare; protein molecular weight standard from Bio-Rad; series of lectin conjugated agarose from Seikagaku Corporation; Aβ40 from Peptide Institute; BCA protein assay reagent and sulfo-NHS-LC-biotin from Thermo Fisher Scientific Inc.; all other chemical products from Sigma or Wako Chemicals. The oligoDNA primer was obtained from Invitrogen. The primers used are shown in Table 1. The anti-APP rabbit polyclonal antibody (C15 antibody) to endogenous membrane-bound (intact) APP was provided by Dr. Kei Maruyama (Saitama Medical University, Saitama, Japan). The commercially available antibodies used were as follows: mouse monoclonal anti-APP 22C11 antibody (Chemicon); anti-human sAPPα antibody (6E10; Signet Laboratories); anti-GAPDH antibody (Chemicon); anti-KPI antibody (Chemicon); anti-OX2 antibody (Chemicon); anti-human sAPPβantibody (IBL Co.); goat polyclonal anti-PECAM antibody (Santa Cruz Biotechnology). The clinical study was approved by the ethical committee of RIKEN and Fukushima Medical University. All animal experiments were performed according to the guideline of animal experiment by RIKEN.

TABLE 1

| primer name | sequence number | sequence |
|---|---|---|
| 1 | 3 | GTCGACATGCTGCCCGGTTTGGCACTGC |
| 2 | 4 | AAGCTTGTTCTGCATCTGCTCAAAGAAC |
| 3 | 5 | AGCTTGACTACAAGGACGACGATGACAAGTGAT |
| 4 | 6 | CTAGATCACTTGTCATCGTCGTCCTTGTAGTCA |
| APP_{S346A}forward | 7 | TGTGGCAGCGCCATGGCCCAAAGTTTACTCAAG |
| APP_{S346A}reverse | 8 | CTTGAGTAAACTTTGGGCCATGGCGCTGCCACA |
| APP_{S348A}forward | 9 | GCGCCATGTCCCAAGCCTTACTCAAGACTACCC |
| APP_{S348A}reverse | 10 | GGGTAGTCTTGAGTAAGGCTTGGGACATGGCGC |
| APPS_{T352A}forward | 11 | CCCAAAGTTTACTCAAGGCCACCCAGGAACCTC |
| APPS_{T352A}reverse | 12 | GAGGTTCCTGGGTGGCCTTGAGTAAACTTTGGG |
| APP_{T353A}forward | 13 | ACTCAAGACTGCCCAGGAACCTCTTGC |
| APP_{T353A}reverse | 14 | GCAAGAGGTTCCTGGGCAGTCTTGAGT |
| APP_{S346,348A}forward | 15 | GCGCCATGGCCCAAGCCTTACTCAAGACTACCC |
| APP_{S346,348A}reverse | 16 | GGGTAGTCTTGAGTAAGGCTTGGGCCATGGCGC |
| APP_{SOX2A}forward | 17 | GTTTACTCAAGGCCGCCCAGGAACCTCTTGC |
| APP_{SOX2A}reverse | 18 | GCAAGAGGTTCCTGGGCGGCCTTGAGTTAAAC |
| APP_{T291,292A}forward | 19 | TGTTAAACTTCCTGCAGCAGCAGCCAGTACCCC |
| APP_{T291,292A}reverse | 20 | GGGGTACTGGCTGCTGCTGCAGGAAGTTTAACA |
| APP_{T651A}forward | 21 | CGACCGAGGACTGGCCACTCGACCAGG |
| APP_{T651A}reverse | 22 | CCTGGTCGAGTGGCCAGTCCTCGGTCG |
| A | 23 | ATGCTGCCCGGTTTGGCACTG |
| B | 24 | CGAGAGGTGTGCTCTGAACAAG |
| C | 25 | CCATGTCCCAAAGTTTACTCAAG |
| D | 26 | CTAGTTCTGCATCTGCTCAAAG |

### (Expression Plasmid and Cell Culture)

Human APP770 FLAG-pEF was constructed by inserting human APP770 sequence amplified by PCR (primers 1 and 2 were used) into SalI and HindIII sites (of vector), and FLAG region fragment was produced by annealing primers 3 and 4 to the HindIII and XbaI sites. ViraPower adenovirus expression system (Invitrogen) was used according to the protocol of the manufacturer to produce recombinant adenovirus retaining human APP-FLAG. Series of APP770-pcDNA3.1 variants were produced using QuickChange Site-Directed Mutagenesis Kit (Stratagene). To produce APP_{OX2All} variant (all potential O-type sugar chain addition sites in OX2 region were deleted), APP_{S346,348A} was constructed first, and thereafter APP_{OX2All} variant was produced using APP_{S346,348A} as a template and APP_{SOX2A} forward and reverse as primers. To produce APP_{All} variant (all reported O-type sugar chain addition sites in whole APP770 region (J Proteome Res 2009 8(2):631-642) were deleted), the present inventors substituted Thr291, Thr292 and Thr651 by Ala using APP_{OX2All} variant as a template, and APP_{T291,292A} forward and reverse and APP_{T651A} forward and reverse as primers. Human brain microvascular endothelial cell (BMEC, Applied Cell Biology Research Institute) was cultured in CS-C complete medium containing or not containing 10% FBS, and used within 4 passages. HUVEC (TaKaRa) was cultured in EMB-2 (TaKaRa) containing 2% FBS and EGM™-2 SingleQuots and used within 4 passages. The primary liver sinusoidal endothelial cell (LSEC) was prepared from the liver of mouse by using CD146 MicroBeads (Miltenyi Biotec) according to the protocol of manufacturer. COS-7 cell was cultured in DMEM containing 10% FBS.

### (Patients and Sample)

A patient (60 years old) who died of nonketotic hyperosmolar coma was adopted for this test. CSF sample was obtained from the patient with AD. This test was approved by the ethical committee of Fukushima Medical University (No. 613).

### (PCR Analysis of APP Transcript)

Using Sepasol reagent (Nacalai Tesque, Inc.), total RNA was isolated from the cell, isolated RNA (5 μg) was reversely transcribed using a random hexamer and Super Script III RT Kit (Invitrogen) according to the manufacturer's protocol. The obtained cDNA samples were subjected to PCR analysis using primers A and D for APP695, APP751 and APP770, primers B and D for APP751 and APP770, and primers C and D for APP770. The PCR was carried out for 28 cycles (95° C. for 40 sec, 56° C. for 40 sec and 72° C. for 90 sec).

### (Immunohistochemistry)

The brain tissue was fixed with a phosphate buffered 15% formalin solution and embedded in Parafilm. One of a pair of serial sections (thickness 5 μm) was stained with hematoxylin and eosin. The other was incubated with anti-OX2 antibody (1:100) at 37° C. overnight, and incubated with biotinylate anti-rabbit IgG (1:200). The bound antigen was visualized by the avidin-biotin-peroxidase complex method (ABC kit; Vector Laboratories).

### (Western Blotting)

The cell lysate was solubilized in a T-PER buffer containing protease inhibitor cocktail (Roche), subjected to SDS-PAGE (5-20% gradient gel), and transferred to a nitrocellulose membrane. Then, the membrane was incubated with anti-APP 22C11 (1:1,000 dilution), anti-APP C15 (1:1,000 dilution), anti-sAPPβ (1:500 dilution), anti-sAPPα (1:1,000 dilution), anti-KPI (1:250 dilution) and anti-OX2 antibody (1:250 dilution). Appropriate horseradish peroxidase-donkey anti-goat IgG (Jackson ImmunoResearch Laboratories), and anti-mouse and anti-rabbit IgG (GE Healthcare) antibody were used as secondary antibodies (1:1,000 dilution). A chemical luminescence substrate (Thermo Fisher Scientific Inc.) was used for detection of the bound antibody. As a loading control, the same membrane was incubated with anti-GAPDH antibody (1:250 dilution; Chemicon) to detect GAPDH. The detected signal was quantified by Luminoimage Analyzer LAS-1000 PLUS (Fujifilm). The medium from BMEC was incubated with SSA- or ConA-agarose for 16 hr. The medium from human CSF (0.2-0.5 ml) or cultured cells was incubated with heparin-sepharose (Thermo Fisher Scientific Inc.). The precipitate was washed 3 times with PBS before SDS-PAGE.

### (Quantification of Aβ)

BMEC was infected with an adenovirus preparation for APP770-FLAG overexpression, and cultured in Opti-MEM for 8 hr. The levels of Aβ40 and Aβ42 in the medium were determined using Human Amyloid β (1-40) or (1-42) Assay Kit (IBL Co.) for BMEC, and Human/Rat β Amyloid (40) or (42) ELISA Kit (Wako Chemicals) for mouse neuron.

(Digestion with Glycosidase)

A sample precipitated from the cell lysate or medium with heparin was incubated in the presence or absence of Arthrobacter ureafaciens sialidase (4 μU) and/or O-glycanase (2 mU) for 18 hr.

### (Biotinylation of Cell Surface)

BMEC grown in a 10 cm culture dish was labeled with Sulfo-NHS-LC-Biotin at 4° C. for 30 min. The cells were washed 3 times with PBS (pH 8.0) containing 0.1M glycine and once with PBS to prepare a cell lysate. For further analysis, the biotinylated cell surface protein was pulled down with streptavidin-sepharose (GE Healthcare).

(Treatment with benzyl GalNAc)

Subconfluent BMEC grown in the 10 cm tissue culture dish was incubated in the presence of benzyl GalNAc (2 mM) for 18 hr. Thereafter, a cell lysate and medium samples were prepared for further analyses.

### Example 1

The present Example explains cell type-specific expression of 3 kinds of isoforms by selective splicing of APP.

While human brain expresses APP mRNA isoforms APP695, APP751 and APP770 by 3 kinds of selective splices (Nature 1988 331(6156):525-527; Nature 1988 331(6156):528-530) (FIG. 1A), neuron expresses only for APP695 (Proc Natl Acad Sci USA 1993 90 (20):9513-9517; Proc Natl Acad Sci USA 1990 87(4):1561-1565; Proc Natl Acad Sci USA 1989 86(16):6338-6342). Such information suggests cell type-specific APP splicing phenomenon occurs in the brain. In the present Example, the present inventors took note of cerebral endothelial cell, and analyzed cerebral endothelial APP using established primary human brain microvascular endothelial cell (BMEC). Western blot analysis using anti-APP C-terminal antibody (C15) has revealed that BMEC expresses APP at a level equivalent to or higher than that of primary neuron (FIG. 1B). Endothelial APP showed two separate bands with different gel mobility after SDS-PAGE. Since two endothelial APPs were detected by both anti-KPI antibody and anti-OX2 antibody, endothelial cell was concluded to express APP770. Since neuron APP was not detected by both anti-KPI antibody and anti-OX2 antibody, the previous report on the expression of only APP695 by neuron (Proc Natl Acad Sci USA 1993 90(20):9513-9517; Proc Natl Acad Sci USA 1990 87(4):1561-1565) was confirmed. When BMEC lysate was analyzed by anti-KPI antibody or anti-OX2 antibody, one additional band was found between two APP bands. This band is highly likely a processed form of APP or sAPP lacking the C-terminal sequence of APP. While human umbilical cord blood-derived vascular endothelial cell (HUVEC) showed a remarkably lower expression level than that of BMEC, it expressed two kinds of the type of APP, thus showing wide expression of APP770 in endothelial cells.

To analyze APP transcript, the present inventors have isolated RNA from the endothelial cells and primary neuron. Then, the reversely transcribed cDNA sample was analyzed by PCR using a series of oligonucleotide primers to detect APP695, APP751 and APP770. As shown in FIG. 1C, the neuron expressed APP695, but the endothelial cell expressed APP770 and did not express APP695. These results have confirmed cell type-specific APP expression.

### Example 2

The present Example explains sugar chain addition state of APP770.

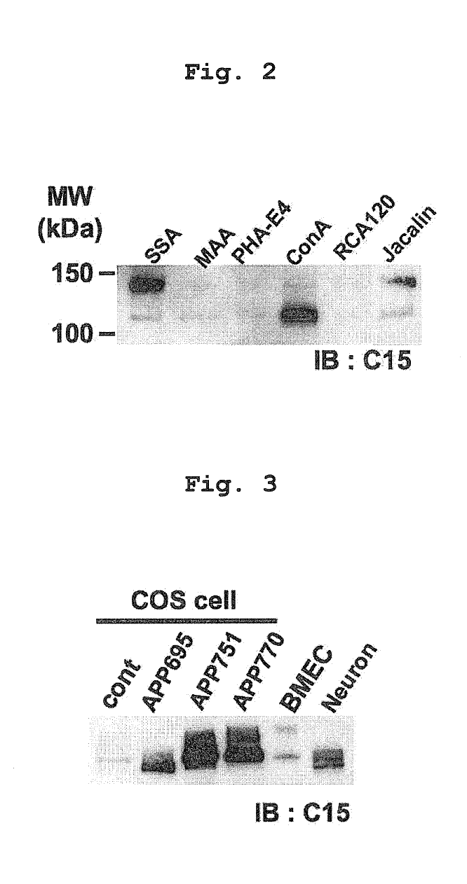

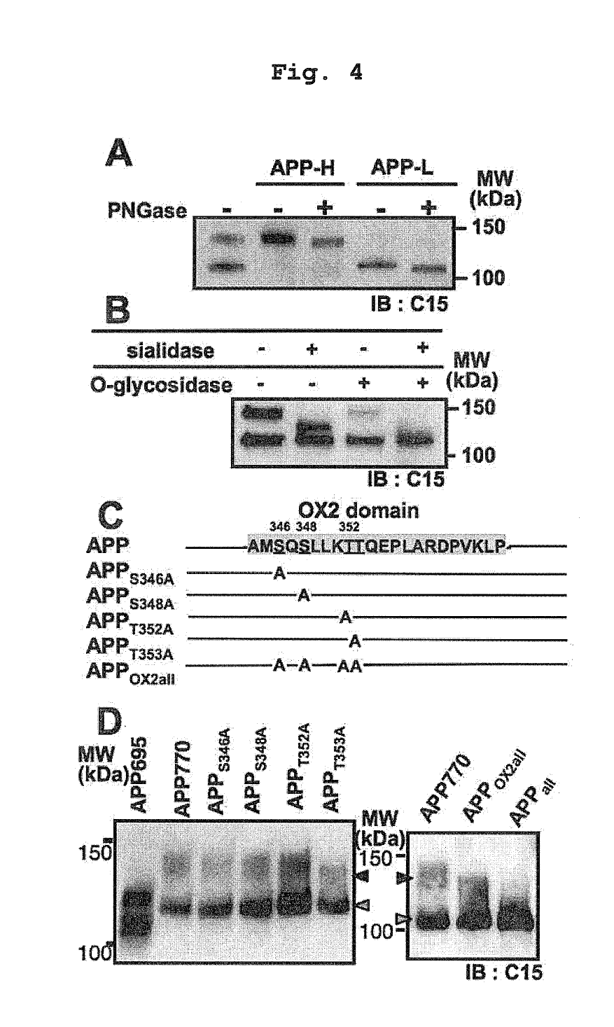

Since APP may contain two N-linked and plural O-linked sugar chains (Biochim Biophys Acta 1999 1472(1-2):344-358; J Proteome Res 2009 8(2):631-642; J Biol Chem 1998 273(11):6277-6284), the present inventors have predicted that APP770 having high molecular weight (APP-H) is highly added with sugar chains. Based on the results of the series of lectin pull down assays (FIG. 2), APP-H and APP770 having low molecular weight (APP-L) were each separated by Sambucus sieboldiana agglutinin (SSA)- and concanavalin A(ConA)-agarose, and digested with peptide N-glycosidase (PNGase) to remove N-linked sugar chain. As shown in FIG. 4A, after the PNGase treatment, the two APP forms both moved slightly faster in SDS-PAGE gel than before the treatment. Therefore, it was shown that the both forms have an N-linked sugar chain, and the difference in the molecular weight of the both forms cannot be explained by N-linked sugar chain alone.

Since APP-H has affinity for SSA lectin that recognizes Siaα2, 6Gal/GalNAc, the present inventors predicted that APP-H contains O-linked sugar chain having a sialic acid residue. After the sialidase treatment, the mobility of APP-H remarkably changed, and became close to that of APP-L (FIG. 4B). After the treatment with sialidase and O-glycosidase, the mobility of APP-H completely matched with that of APP-L. Since O-glycosidase cleaves unsubstituted core-1 type O-linked sugar chain alone, the present inventors have concluded that APP-H has a sialylated core-1 type O-linked sugar chain. When APP695, APP751 or APP770 was overexpressed in COS cells, the difference in the mobility between two forms of APP was always observed clearly for APP770 (FIG. 3). The data suggest the presence of O-linked sugar chain in OX2 region.

Thus, the present inventors have produced a series of APP770 variants wherein each Ser/Thr residue in the OX2 region was individually substituted by Ala (FIG. 4C). Then, wild-type APP770 and a variant thereof were overexpressed in COS cells. Although an O-linked sugar chain addition mechanism is not sufficiently developed in COS cells, the present inventors have detected APP-H at a low level (FIG. 4D). While mutation in Ser346, Ser348 and Thr352 did not affect the mobility of APP variant on SDS-PAGE gel, APP_{T353A} variant (Thr353 was substituted to Ala) moved faster than the wild-type APP (FIG. 4D). The results suggest that the O-linked sugar chain is added to Thr353.

The finding that both APP_{T353A} and APP_{OX2all} (wherein all Ser/Thr residues in OX2 region were mutated to Ala) still showed two separate bands suggests further presence of an O-linked sugar chain addition site in a region other than the OX2 region of APP770. Perdivara et al. (J Proteome Res 2009 8(2):631-642) have recently showed that APP695 is modified with O-linked sugar chain in Thr291, Thr292 and Thr576. Thus, the present inventors have now produced APP770 variant APPₐₗₗ (which is APP_{OX2all} wherein Thr291, Thr292, Thr651 (Thr576 for APP695) and Thr652 were further substituted by Ala). The APPₐₗₗ all showed an almost single band (FIG. 4D), which indicates that the present inventors mutated all O-linked sugar chain addition sites of APP770 in the APPₐₗₗ variant.

### Example 3

The present Example explains sugar chain addition state of sAPP secreted from brain microvascular endothelial cell BMEC.

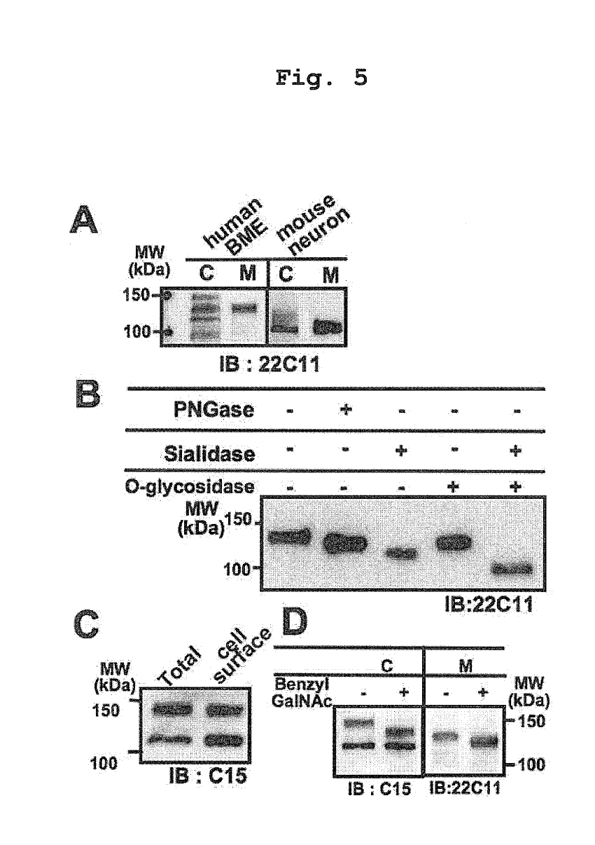

To analyze APP metabolite in endothelial cells, the present inventors performed Western blot analysis of BMEC lysate and medium samples by using an anti-APP 22C11 antibody (which recognizes APP N-terminal region). As shown in FIG. 5A, a single sAPP band having mobility between two bands of APP (APP-H and APP-L) found in the cell lysate was detected in the medium. This suggests that sAPP is derived solely from APP-H. In fact, sAPP is sensitive to both sialidase and O-glycosidase treatments (FIG. 5B), and this shows that sAPP contains sialylated core-1 type O-linked sugar chain.

It is noteworthy that the present inventors could not detect sAPP without an O-linked sugar chain. In consideration of the general aspect that cleavage of APP at the α site seems to occur on the cell surface, whereas that at the β site occurs in the endocytosis pathway (J Cell Biol 2003 160(1):113-123), a possibility was also considered that APP without O-linked sugar chain addition cannot move to the cell surface, and cannot contact any of α-secretase and β-secretase. However, the cell surface biotinylation experiment has revealed that APP-H and APP-L both reach the cell surface (FIG. 5C). Furthermore, benzyl GalNAc (inhibitor of O-linked sugar chain chain elongation) did not inhibit secretion of sAPP into the medium (FIG. 5D). These suggest that addition of one GalNAc to APP770 is sufficient for APP770 to enter the secretion pathway.

### Example 4

The present Example explains that APP770 is expressed in human cerebral blood vessel, cleaved at the α and β sites, and secreted into CSF.

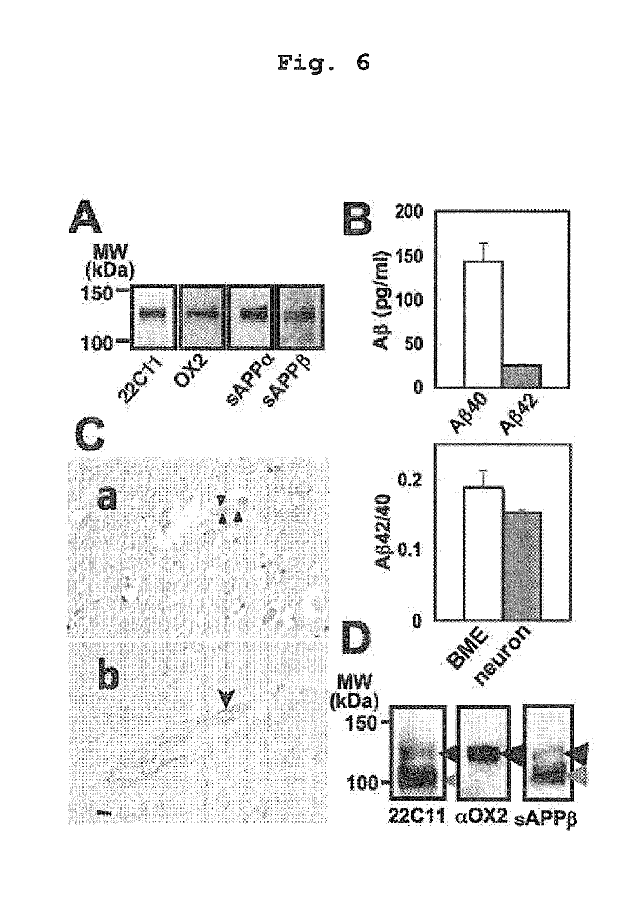

As for APP770 processing, since only limited information is available as to the expression levels and activities of endothelial α secretase and β secretase, the present inventors have analyzed soluble sAPP770 secreted from BMEC. When a specific antibody to sAPPα and sAPP used, sAPPα and sAPP13 were both detected (FIG. 6A).

Since endothelial cell has an amyloid-producing β secretase pathway, these cells can be considered to also have γ secretase activity and produce Aβ peptide. Both Aβ40 and Aβ42 were detected in a medium of BMEC that overexpresses APP770, and the ratio of endothelial Aβ42/Aβ40 was similar to that in neuron (FIG. 6B).

To clarify whether APP770 is actually expressed in cerebral blood vessel, the present inventors have first analyzed a cerebral cortex section by using an anti-OX2 antibody, and determined the localization of APP770. While the luminal region of endothelial cells of the vein and venule was stained with anti-OX2 antibody, smooth muscle cell was not stained (FIG. 6C). In arachnoid blood vessel, immunohistochemical signal was not observed.

Next, whether CSF contains sAPP770β (β secretase cleavage product on APP770 N-terminal side) was examined. sAPP was pulled down from CSF by using heparin-agarose, and immunostaining was performed using anti-APP22C11 antibody, anti-OX2 antibody and anti-sAPPβantibody. Two bands were detected by the anti-APP22C11 antibody, of which only the upper band was detected by the anti-OX2 antibody (FIG. 6D). This means that the upper band was derived from APP770. Detection of both the upper and lower bands with the anti-sAPPβ antibody means that the both forms contained β secretase cleavage product. These results indicate that CSF obtained from the subject can be adopted as a sample for examining the presence or absence of sAPP770β production by endothelial cells in the body.

### INDUSTRIAL APPLICABILITY

The present invention enables detection of soluble amyloid β precursor protein 770β derived from endothelial cell. The obtained data can be utilized for diagnosing a disease accompanied by accumulation of amyloid β peptide such as Alzheimer's disease and the like. In addition, using the diagnostic reagent and diagnosis kit of the present invention, whether being affected with a disease accompanied by accumulation of amyloid β peptide can be conveniently diagnosed with high accuracy.

This application is based on a patent application No 2010-171122 filed in Japan (filing date: Jul. 29, 2010), the contents of which are incorporated in full herein.

1. A method of detecting soluble amyloid β precursor protein 770β derived from vascular endothelial cell, comprising the following steps:
(1) a step of contacting a biological sample derived from a test subject with an antibody recognizing soluble amyloid β precursor protein β or an amyloid β precursor protein 770-specific antibody; and
(2) a step of detecting soluble amyloid β precursor protein 770β in the complex formed in step (1).

2. (canceled)

3. The method according to claim 1, wherein the biological sample is blood, plasma or serum.

4. The method according to claim 1, wherein the biological sample is cerebrospinal fluid.

5. The method according to claim 1, wherein the biological sample is culture supernatant of vascular endothelial cell.

6. The method according to claim 1, comprising, in addition to steps (1) and (2), detection of soluble amyloid β precursor protein 695β by the following steps:
(3) a step of contacting a biological sample derived from the test subject suspected of having a disease accompanied by accumulation of amyloid β peptide, with an antibody recognizing soluble amyloid β precursor protein β or soluble amyloid β precursor protein 695β-specific antibody; and
(4) a step of detecting soluble amyloid β precursor protein 695β in the complex formed in step (3).

7. The method according to claim 6, wherein, in step (3), one of the antibody recognizing soluble amyloid β precursor protein β and the amyloid β precursor protein 695-specific antibody is used, and
step (4) comprises
(4a) a step of contacting soluble amyloid β precursor protein 695β in the complex formed in step (3), with the antibody recognizing soluble amyloid β precursor protein β or soluble amyloid β precursor protein 695β-specific antibody, which was not used in step (3); and
(4b) a step of detecting the complex formed in step (4a).

8. The method according to claim 6, wherein the biological sample in step (1) is blood, plasma, serum, culture supernatant of vascular endothelial cell or cerebrospinal fluid, and the biological sample in step (3) is cerebrospinal fluid.

9. The method according to claim 1, wherein the amyloid β precursor protein 770-specific antibody is an antibody recognizing OX2 domain.

10. The method according to claim 1, wherein the test subject is suspected of having a disease accompanied by accumulation of amyloid β peptide.

11. The method according to claim 10, wherein the disease is at least any one of cerebrovascular amyloid angiopathy, cerebrovascular dementia, cerebral infarction and Alzheimer's disease.

12. The method according to claim 1, wherein the test subject is suspected of having Alzheimer's disease occurring in association with at least any one of cerebrovascular amyloid angiopathy, cerebrovascular dementia and cerebral infarction.

13.-17. (canceled)

18. A method of detecting soluble amyloid β precursor protein 770β derived from vascular endothelial cell, comprising the following steps:
(1) a step of contacting a biological sample derived from a test subject with an antibody recognizing soluble amyloid β precursor protein β or an amyloid β precursor protein 770-specific antibody;
(2a) a step of contacting soluble amyloid β precursor protein 770β in the complex formed in step (1) with the antibody recognizing soluble amyloid β precursor protein β or amyloid β precursor protein 770-specific antibody, which was not used in step (1); and
(2b) a step of detecting the complex formed in step (2a).

19. The method according to claim 18, wherein the biological sample is blood, plasma, serum, cerebrospinal fluid or culture supernatant of vascular endothelial cell.

20. The method according to claim 18, comprising, in addition to steps (1), (2a) and (2b), detection of soluble amyloid β precursor protein 695β by the following steps:
(3) a step of contacting a biological sample derived from the test subject suspected of having a disease accompanied by accumulation of amyloid β peptide, with an antibody recognizing soluble amyloid β precursor protein β or soluble amyloid β precursor protein 695β-specific antibody;
(4a) a step of contacting soluble amyloid β precursor protein 695β in the complex formed in step (3), with the antibody recognizing soluble amyloid β precursor protein β or soluble amyloid β precursor protein 695β-specific antibody, which was not used in step (3); and
(4b) a step of detecting the complex formed in step (4a).

21. The method according to claim 20, wherein the biological sample in step (1) is blood, plasma, serum, culture supernatant of vascular endothelial cell or cerebrospinal fluid, and the biological sample in step (3) is cerebrospinal fluid.

22. The method according to claim 18, wherein the amyloid β precursor protein 770-specific antibody is an antibody recognizing OX2 domain.

23. The method according to claim 18, wherein the test subject is suspected of having a disease accompanied by accumulation of amyloid β peptide.

24. The method according to claim 23, wherein the disease is at least any one of cerebrovascular amyloid angiopathy, cerebrovascular dementia, cerebral infarction and Alzheimer's disease.

25. The method according to claim 18, wherein the test subject is suspected of having Alzheimer's disease occurring in association with at least any one of cerebrovascular amyloid angiopathy, cerebrovascular dementia and cerebral infarction.


SEQUENCE LISTING (26 sequences)

SEQ ID NO: 1 - DNA, 3641 bp, Homo sapiens
gctgactcgcctggctctgagccccgccgccgcgctcgggctccgtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaacttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagcaaaaaaaaaaaaaaa

SEQ ID NO: 2 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 3 - DNA, 28 bp, Artificial Sequence
  PCR primer
gtcgacatgctgcccggtttggcactgc

SEQ ID NO: 4 - DNA, 28 bp, Artificial Sequence
  PCR primer
aagcttgttctgcatctgctcaaagaac

SEQ ID NO: 5 - DNA, 33 bp, Artificial Sequence
  PCR primer
agcttgactacaaggacgacgatgacaagtgat

SEQ ID NO: 6 - DNA, 33 bp, Artificial Sequence
  PCR primer
ctagatcacttgtcatcgtcgtccttgtagtca

SEQ ID NO: 7 - DNA, 33 bp, Artificial Sequence
  PCR primer
tgtggcagcgccatggcccaaagtttactcaag

SEQ ID NO: 8 - DNA, 33 bp, Artificial Sequence
  PCR primer
cttgagtaaactttgggccatggcgctgccaca

SEQ ID NO: 9 - DNA, 33 bp, Artificial Sequence
  PCR primer
gcgccatgtcccaagccttactcaagactaccc

SEQ ID NO: 10 - DNA, 33 bp, Artificial Sequence
  PCR primer
gggtagtcttgagtaaggcttgggacatggcgc

SEQ ID NO: 11 - DNA, 33 bp, Artificial Sequence
  PCR primer
cccaaagtttactcaaggccacccaggaacctc

SEQ ID NO: 12 - DNA, 33 bp, Artificial Sequence
  PCR primer
gaggttcctgggtggccttgagtaaactttggg

SEQ ID NO: 13 - DNA, 27 bp, Artificial Sequence
  PCR primer
actcaagactgcccaggaacctcttgc

SEQ ID NO: 14 - DNA, 27 bp, Artificial Sequence
  PCR primer
gcaagaggttcctgggcagtcttgagt

SEQ ID NO: 15 - DNA, 33 bp, Artificial Sequence
  PCR primer
gcgccatggcccaagccttactcaagactaccc

SEQ ID NO: 16 - DNA, 33 bp, Artificial Sequence
  PCR primer
gggtagtcttgagtaaggcttgggccatggcgc

SEQ ID NO: 17 - DNA, 31 bp, Artificial Sequence
  PCR primer
gtttactcaaggccgcccaggaacctcttgc

SEQ ID NO: 18 - DNA, 31 bp, Artificial Sequence
  PCR primer
gcaagaggttcctgggcggccttgagtaaac

SEQ ID NO: 19 - DNA, 33 bp, Artificial Sequence
  PCR primer
tgttaaacttcctgcagcagcagccagtacccc

SEQ ID NO: 20 - DNA, 33 bp, Artificial Sequence
  PCR primer
ggggtactggctgctgctgcaggaagtttaaca

SEQ ID NO: 21 - DNA, 27 bp, Artificial Sequence
  PCR primer
cgaccgaggactggccactcgaccagg

SEQ ID NO: 22 - DNA, 27 bp, Artificial Sequence
  PCR primer
cctggtcgagtggccagtcctcggtcg

SEQ ID NO: 23 - DNA, 21 bp, Artificial Sequence
  PCR primer
atgctgcccggtttggcactg

SEQ ID NO: 24 - DNA, 22 bp, Artificial Sequence
  PCR primer
cgagaggtgtgctctgaacaag

SEQ ID NO: 25 - DNA, 23 bp, Artificial Sequence
  PCR primer
ccatgtcccaaagtttactcaag

SEQ ID NO: 26 - DNA, 22 bp, Artificial Sequence
  PCR primer
ctagttctgcatctgctcaaag